(19) United States
(12) Patent Application Publication
(10) Pub. No.: US 2009/0208542 A1
(43) Pub. Date: Aug. 20, 2009

(54) PRODUCT AND METHOD FOR TRANSFERRING HEATED FLOWABLE COSMETIC ONTO SKIN

(75) Inventor: Stephen Roy Barrow, Trumbull, CT (US)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., d/b/a UNILEVER, Englewood Cliffs, NJ (US)

(21) Appl. No.: 12/032,731

(22) Filed: Feb. 18, 2008

(51) Int. Cl. A61K 8/02; A61Q 99/00

(52) U.S. Cl. 424/401

(57) ABSTRACT

A product and method for treating skin with a heated lotion is herein described. The product is a packaged cosmetic composition with associated instructions for applying to skin a heated form of the cosmetic composition dispensed from a heating device. The cosmetic composition includes porous particles which function to retain heat within the composition to a greater extent than in the absence of the particles.

### BACKGROUND OF THE INVENTION

1. Field of the Invention

The invention concerns a cosmetic product that includes a flowable cosmetic composition dispensed from a heating device wherein the composition has extended heat retention.

2. The Related Art

Heated lotions have several benefits. A fleeting unpleasant wince may occur when a cold fluid is first topically applied to human skin. Warmed lotions do not elicit the same negative response.

Warmed cosmetic fluids are believed to better penetrate the skin. Warmth is viewed as an assistance in opening pores. This allows deeper penetration into the skin of the cosmetic fluid.

Therapeutic effects may also be achievable by heated lotions. These may mitigate joint aches, sore muscles and other body tightness. For all these reasons, mechanical devices have been developed to heat cosmetic fluids.

U.S. Pat. No. 6,216,911 B1 (Kreitemier et al.) describes an apparatus for quickly heating a predetermined volume of viscous fluid. The fluid is then efficiently dispensed at one or more selected temperatures. In one embodiment, the predetermined volume of viscous fluid is partially housed in a predelivery chamber separate from the main fluid reservoir. An apparatus described by this patent is commercially available from New Sensations LLC, Englewood, Colo. under the brand New Sensation Lotion Spa.

U.S. Patent Application Publication 2002/0108965 A1 (Hill et al.) discloses a fluid heating and dispensing device with a first reservoir, a second reservoir, a pump, a heating device and a delivery device. This document appears to describe a commercially available apparatus from Conair Corporation under the designation HLD 31 and HLD 20.

U.S. Pat. No. 6,056,160 (Carlucci et al.) reports a device for heating and dispensing, to a user through an outlet, a foaming liquid such as shaving cream from a pressurized can.

With the advent of suitable delivery devices, a need has developed to improve the cosmetic fluids dispensed therefrom. An important problem to be solved is the extension of the heating effect for the cosmetic composition subsequent to being dispensed.

### SUMMARY OF THE INVENTION

A cosmetic product for use with a heating device is provided which includes:
(i) a cosmetic composition having from about 0.01 to about 20% by weight of porous particles having an average particle size ranging from about 0.1 to about 100 μm in a cosmetically acceptable carrier;
(ii) a package containing the cosmetic composition; and
(iii) instructions associated with the package describing use of the cosmetic composition which includes charging the cosmetic composition into the heating device, applying heat to the composition, thereafter activating a dispensing mechanism associated with the device and transferring dispensed heated composition to a human body.

A method for treating skin is provided which includes:
(i) providing a cosmetic composition including from about 0.01 to about 20% by weight of porous particles having an average particle size ranging from about 0.1 to about 100 μm in a cosmetically acceptable carrier;
(ii) providing a heating device including a chamber for receiving a flowable cosmetic composition, a heating element for imparting heat to the cosmetic composition, and an outlet for dispensing heated cosmetic composition; and
(iii) applying dispensed heated cosmetic composition onto the skin.

### DETAILED DESCRIPTION OF THE INVENTION

Now it has been found that a heated flowable cosmetic composition can longer retain heat when formulated with even a small amount of porous particles. Upon being placed onto skin, the porous particle fortified compositions maintain warmth on the skin for the critical few seconds to minutes after initial application.

Dispensing devices for heating cosmetic fluid compositions as noted above have been described in U.S. Pat. No. 6,216,911 B1; U.S. Patent Application Publication 2002/0108965 A1 and U.S. Pat. No. 6,056,160, the specifications of which are herein incorporated by reference. Also there are commercial devices available. One device is sold by Conair Corporation of Stamford, Conn. and another by New Sensations LLC of Englewood, Colo.

Heating devices of the present invention are best operated to deliver a composition that exhibits a dispensed temperature between about 30° to about 60° C., more preferably from 36° C. to 54° C., even more preferably from 380 to 49° C. and optimally from 40° to 46° C.

Porous particles of the present invention in preferred embodiments may allow a 10 gram dispensed unit of cosmetic composition to maintain an elevated temperature relative to a composition absent those particles. The elevated temperature may range from 1.50 to 10° C., preferably from 2° to 8° C., optimally from 3° to 6° C. dependent on the level of porous particles formulated into the composition. Maintenance of the elevated temperature may range from 5 seconds to about 10 minutes, preferably from 15 seconds to 5 minutes, more preferably from 10 seconds to 2 minutes, and optimally from 20 seconds to 1 minute upon skin after being dispensed.

Porous particles of the present invention may either be organic or inorganic. Preferably but not necessarily, the particles may be formed as spherical beads. The particles preferably are water-insoluble and polymeric.

By the term “porous” is meant an open or closed cell structure. Preferably the particles are hollow beads. Average particle size may range from about 0.1 to about 100, preferably from about 0.5 to about 50, more preferably from 1 to about 15, optimally from about 3 to about 10 μm.

Representative inorganic materials of this invention are silicas such as Spheron® P-1500 sold by Presperse Inc. However, organic polymers or copolymers are the preferred materials. These can be formed from monomers including the acid, salt or ester forms of acrylic acid, methacrylic acid, methyl methacrylate, ethylacrylate, ethylene, propylene, vinylidene chloride, acrylonitrile, maleic acid, vinyl pyrrolidone, styrene, divinylbenzene, butadiene and mixtures thereof. The polymers are especially useful in cross-linked form. Cells of the porous particles may be filled by a gas which can be air, nitrogen or a hydrocarbon. Oil Absorbance (castor oil) is a measure of porosity and may range from about 10 to about 500, preferably from about 80 to about 300, optimally from about 120 to about 180 mI/100 grams. Density of the particles in preferred embodiments may range from about 0.08 to 0.55, preferably from about 0.15 to 0.48 g/cm³.

Silicone based materials may be employed as the porous particles, most especially spherical organic silicone particles. These may be chosen from microbeads of methylsilsesquioxane resins. Commercially they are available from Toshiba Silicone under the name Tospearl® 145A. Also suitable are spherical particles of cross-linked polydimethylsiloxanes commercially available from the Dow Corning Toray Silicone Company under the name Trefil E-506C® or Trefil E-505C®.

Non-silicone based porous particles may also be suitable. These include microbeads of poly(methyl methacrylate) such as those sold by Seppic under the name of Micropearl M 100®. Spherical particles of polyamide may also be useful, especially those of Nylon 12 such as those sold by Atochem under the name Orgasol 2002 D Nat C05®. Polystyrene microspheres might also be suitable and are sold by Dyno Particles Company under the name Dynospheres®. Styrene/acrylate copolymer particles also have suitability and are available from Rohm & Haas under the name Ropaque™.

Most preferred for this invention are porous polymers of cross-linked poly(methylmethacrylate). Representative is a poly(methylmethacrylate) named as Ganzpearl GMP 0820® available from Presperse, Inc., Piscataway, N.J., known also by its INCI name of Methyl Methacrylate Crosspolymer. The product specifications of Ganzpearl GMP 0820® include: spherical, white fine powder having a particle size of 4-10.5 μm, preferably 4-8 μm, high oil absorption, and specific gravity of 1.10 to 1.25. Its loss on ignition (400° C.) is less than 0.1%, and on drying (105° C./2 hours) is less than 2.0%. The surface residual monomer content of Ganzpearl GMP 0820® is less than 20 ppm, with total residual monomer content being less than 100 ppm.

Methyl methacrylate crosspolymers are also commercially available from Nihon Junyaku under the trademark Jurymer MP-1P and from Tomer under the trademark Microsphere M-305.

Amounts of the porous particles may range from about 0.01 to about 20%, preferably from about 0.1 to about 10%, more preferably from about 0.25 to about 8%, still more preferably from about 0.5 to about 3%, and optimally from about 0.75 to about 2% by weight of the composition.

Compositions of the present invention will also include a cosmetically acceptable carrier. Water is the most preferred carrier. Amounts of water may range from about 1 to about 99%, preferably from about 5 to about 90%, more preferably from about 35 to about 70%, optimally between about 40 and about 60% by weight. Ordinarily the compositions will be water and oil emulsions of the W/O or O/W variety.

Other cosmetically acceptable carriers may include mineral oils, silicone oils, synthetic or natural esters, fatty acids and alcohols and humectants. Amounts of these materials may range from about 0.1 to about 50%, preferably from about 0.1 to about 30%, more preferably from about 1 to about 20% by weight of the composition.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from about 3 to about 9, preferably from about 4 to about 5, silicon atoms.

Linear volatile silicone materials generally have viscosities less than about 5 centistokes at 25° C. while cyclic materials typically have viscosities of less than about 10 centistokes.

Nonvolatile silicone oils useful as carrier material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 100,000 centistokes at 25° C.

Among suitable esters are:
(1) Alkenyl or alkyl esters of fatty acids having 10 to 20 carbon atoms, Examples thereof include isopropyl palmitate, isopropyl isostearate, isononyl isonanonoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxy-ethylene sorbitan fatty acid esters are satisfactory polyhydric alcohol esters.
(4) Wax esters such as beeswax, spermaceti, myristyl myristate, stearyl stearate.
(5) Sterols esters, of which soya sterol and cholesterol fatty acid esters are examples thereof.

Fatty acids having from 10 to 30 carbon atoms may be included in the compositions of this invention. Illustrative of this category are Ipelargonlc, lauric, myristic, palmitic, stearic, isostearic, hydroxyystearic, oleic, linoleic, ricinoleic, arachidic, behenic and erucic acids.

Humectants of the polyhydric alcohol-type may also be included in the compositions of this invention. The humectant aids in increasing the effectiveness of the emollient, reduces scaling, stimulates removal of built-up scale and improves skin feel. Typical polyhydric alcohols include glycerol (also known as glycerin), polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably glycerin. The amount of humectant may range anywhere from 0.5 to 30%, preferably between 1 and 15% by weight of the composition.

Emulsifiers may be present in cosmetic compositions of the present invention. Total concentration of the emulsifier may range from about 0.1 to about 40%, preferably from about 1 to about 20%, optimally from about 1 to about 5% by weight of the total composition. The emulsifier may be selected from the group consisting of anionic, nonionic, cationic and amphoteric actives. Particularly preferred nonionic surfactants are those with a C₁₀-C₂₀ fatty alcohol or acid hydrophobe condensed with from about 2 to about 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di- C₈-C₂₀ fatty acids; and polyoxyethylene sorbitan as well as combinations thereof. Alkyl polyglycosides and saccharide fatty amides (e.g. methyl gluconamides) are also suitable nonionic emulsifiers.

Preferred anionic emulsifiers include soap, alkyl ether sulfate and sulfonates, alkyl sulfates and sulfonates, alkylbenzene sulfonates, alkyl and dialkyl sulfosuccinates, C₈-C₂₀ acyl isethionates, C₈-C₂₀ alkyl ether phosphates, alkylethercarboxylates and combinations thereof.

Preservatives can desirably be incorporated into the cosmetic compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are iodopropynyl butyl carbamate, phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients in the emulsion. Preservatives are preferably employed in amounts ranging from about 0.01% to about 2% by weight of the composition.

Thickening agents may be included in compositions of the present invention. Particularly useful are the polysaccharides. Examples include starches, natural/synthetic gums and cellulosics. Representative of the starches are chemically modified starches such as aluminum starch octenylsuccinate. Suitable gums include xanthan, sclerotium, pectin, karaya, arabic, agar, guar, carrageenan, alginate and combinations thereof, Suitable cellulosics include hydroxypropyl cellulose, hydroxypropyl methylcellulose, ethylcellulose and sodium carboxy methylcellulose. Synthetic polymers are still a further class of effective thickening agent. This category includes crosslinked polyacrylates such as the Carbomers, polyacrylamides such as Sepigel® 305 and taurate copolymers. Particularly preferred are the taurate copolymers such as Acryloyl Dimethyltaurate/Vinyl Pyrrolidone Copolymer (available commercially as Aristoflex® AVC) and Sodium Acrylate/Acryloyldimethyl Taurate Copolymer (available commercially as Simulgel EG).

Amounts of the thickener may range from about 0.001 to about 5%, preferably from about 0.1 to about 2%, optimally from about 0.2 to about 0.5% by weight.

Colorants, fragrances and abrasives may also be included in compositions of the present invention. Each of these substances may range from about 0.05 to about 5%, preferably between 0.1 and 3% by weight.

Except in the operating and comparative examples, or where otherwise explicitly indicated, all numbers in this description indicating amounts of material ought to be understood as modified by the word “about”.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

The following Examples will more fully illustrate the embodiments of this invention. All parts, percentages and proportions referred to herein and in the appended claims are by weight unless otherwise indicated.

### EXAMPLE 1

A typical emulsion type cosmetic composition of the present invention is reported in Table 1.

TABLE 1

| Component | Weight % |
|---|---|
| Glycerin | 10.00 |
| Stearic Acid | 2.34 |
| Glyceryl Monostearate/Stearamide AMP | 1.38 |
| Isopropylmyristate | 1.30 |
| Petrolatum | 1.25 |
| Silicone 50 ct | 1.00 |
| Simulgel EG ® | 0.75 |
| Triethanolamine (99%) | 0.70 |
| Glycerol Monostearate | 0.64 |
| Ganzpearl GMP 0820 ® | 0.50 |
| Cetyl Alcohol | 0.37 |
| Fragrance | 0.30 |
| DMDM Hydantoin | 0.17 |
| Titanium Dioxide | 0.10 |
| Glydant Plus ® | 0.09 |
| Disodium EDTA | 0.05 |
| Water | Balance |

The composition of Table 1 was formulated in the following manner. A reactor was charged with the deionized water and disodium EDTA. Heat was applied till 60° C. in combination with stirred mixing. Simulgel EG® was added to the reactor and the temperature maintained at 77-80° C. for 10 to 15 minutes. In a separate vessel, the oil phase components were added. Light mixing of the batch was performed with heating in a water bath to 75-77° C. The water reactor was maintained at 60-65° C. and slow addition occurred for glycerin, titanium dioxide and triethanolamine. Continuous mixing was done until the aqueous system was uniform. Very slowly the oil phase was added to the water phase at 75-77° C. under moderate mixing. After full emulsification, the batch was agitated for a further 5 minutes. Thereupon the resultant emulsion was homogenized using an ARDE Barenco® apparatus for 20-30 seconds at 35%. The resultant system was then topped with further deionized water. Cooling then began with a large sweep (50 rpm) mixer. Preservatives Glydant Plus® and DMDM Hydantoin were then added with the batch held at 50-55° C. Thereafter a slurry of Ganzpearl GMP 0820® in the silicone oil was added to the batch. At a temperature of 45-50° C., the fragrance was charged to the reactor. Heating was then discontinued and mixing was stopped when the temperature reached 38-40° C.

After formation, the composition was charged into a Conair HLD31® Hot Lotion Dispenser. An essentially identical composition but without the porous cross-linked poly(methlymethacrylate), Ganzpearl GMP 0820®, was prepared in parallel and placed into a separate Conair HLD31® Hot Lotion Dispenser. Approximately a 10 gm amount of each of the compositions was dispensed from the Conair appliance onto left and right hands, respectively of several evaluators. The composition that included Ganzpearl® noticeably retained heat longer than the control sample without the Ganzpearl®.

### EXAMPLE 2

Experiments were conducted to quantitivately evaluate the heat retention effect of Ganzpearl® in formulas according to the present invention in the context of dispensing from a heated lotion appliance. A series of five formulas with different levels of Ganzpearl® were evaluated. These formulas utilized the composition of Example 1, except for variation in the level of Ganzpearl®.

Time to cool subsequent to dispensing from the hot lotion appliance was measured according to the following procedure. A liquid crystal film sourced from Educational Innovations Inc., Norwalk, Conn. served as the temperature sensor. This film displays colors as a function of temperature between 30 and 35° C. This temperature range was selected based on typical skin surface temperatures of 32°. A Gardener Wet Film Applicator Rod (No. 22) was employed to create a uniform wet film (56.4 micron thickness) of each sample lotion on the liquid crystal film. The color of the film was recorded each minute until the film surface had cooled below 30° C. The color was then converted into temperature values with a color scale obtained by calibrating the film's color in an oven.

Samples were run side-by-side. Always the sample without Ganzpear® was placed adjacent those with varying levels of this material. One side of the film, attached to a board to facilitate the casting process, consistently cooled faster. This was measured in use to normalize the data against baseline. Table 2 summarizes the results showing effects of the addition of Ganzpearl® on the time to cool from 35° C. to 30° C.

TABLE 2

| Weight % Ganzpearl | % Increase In Time To Cool |
|---|---|
| 0.00 | — |
| 0.25 | 6 |
| 0.50 | 32 |
| 0.75 | 81 |
| 1.00 | 87 |

Evident from the results is that even small amounts of Ganzpearl® had a significant effect upon increasing the “time to cool”. After 1% Ganzpearl®, the effect appears to plateau.

What is claimed is:

1. A cosmetic product for use with a heating device comprising:
(i) a cosmetic composition comprising from about 0.01 to about 20% by weight of porous particles having an average particle size ranging from about 0.1 to about 100 μm in a cosmetically acceptable carrier;
(ii) a package containing the cosmetic composition; and
(iii) instructions associated with the package describing use of the cosmetic composition which comprises charging the cosmetic composition into the heating device, applying heat to the composition, activating a dispensing mechanism associated with the device and transferring dispensed heated composition to a human body.

2. The product according to claim 1 wherein the porous particles are spherical beads.

3. The product according to claim 1 wherein the porous particles have an average particle size ranging from about 0.1 to about 10 μm.

4. The product according to claim 1 wherein the porous particles are cross-linked materials.

5. The product according to claim 1 wherein the porous particles are polymers formed from monomers including the acid, salt or ester forms of acrylic acid, methacrylic acid, methyl methacrylate, ethylacrylate, methylmethacrylate, ethylene, propylene, vinylidene chloride, acrylonitrile maleic acid, vinylpyrrolidone, styrene, divinylbenzene, butadiene and mixtures thereof.

6. The product according to claim 1 wherein the porous particles have an Oil Absorbance ranging from about 10 to about 500 ml/100 grams.

7. The product according to claim 1 wherein the porous particles are cross-linked poly(methylmethacrylate).

8. A method for treating skin comprising:
(i) providing a cosmetic composition comprising from about 0.01 to about 20% by weight of porous particles having an average particle size ranging from about 0.1 to about 100 μm in a cosmetically acceptable carrier;
(ii) providing a heating device comprising a chamber for receiving a flowable cosmetic composition, a heating element for imparting heat to the cosmetic composition, and an outlet for dispensing heated cosmetic composition; and
(iii) applying dispensed heated cosmetic composition onto the skin.

9. The method according to claim 8 wherein the porous particles are spherical beads.

10. The method according to claim 8 wherein the porous particles have an average particle size ranging from about 0.1 to about 10 μm.

11. The method according to claim 8 wherein the porous particles are cross-linked materials.

12. The method according to claim 8 wherein the porous particles are polymers formed from monomers including the acid, salt or ester forms of substances selected from group consisting of acrylic acid, methacrylic acid, methyl methacrylate, ethylacrylate, methylmethacrylate, ethylene, propylene, vinylidene chloride, acrylonitrile maleic acid, vinylpyrrolidone, styrene, divinylbenzene, butadiene and mixtures thereof.

13. The method according to claim 8 wherein the porous particles have an Oil Absorbance ranging from about 10 to about 500 ml/100 grams.

14. The method according to claim 8 wherein the porous particles are cross-linked poly(methylmethacrylate).

15. The method according to claim 8 wherein the cosmetic composition is contained in a package, and the method further comprises associating instructions with the package describing use of the cosmetic composition which comprises charging the cosmetic composition into the heating device, applying heat to the composition, activating a dispensing mechanism associated with the device and transferring dispensed heated composition to a human body.
